(19) United States
(12) Patent Application Publication
(10) Pub. No.: US 2020/0125647 A1
(43) Pub. Date: Apr. 23, 2020

(54) DETERMINATION OF BIORHYTHMS THROUGH VIDEO JOURNAL SERVICES

(71) Applicant: International Business Machines Corporation, Armonk, NY (US)

(72) Inventor: Daphne Mintz, Atlanta, GA (US)

(21) Appl. No.: 16/168,622

(22) Filed: Oct. 23, 2018

(51) Int. Cl. G06F 17/30; G06K 9/00; A61B 5/16; G06Q 50/00

(52) U.S. Cl. CPC G06F 17/30035; G06K 9/00744; G06Q 50/01; A61B 5/167; G06K 9/00335

(57) ABSTRACT

A video journal service which captures short video segments of the user throughout the day for extended periods (day, weeks, or months). The collections of the short video segments are used to build a user profile that can be used by a services that rely on personality or behavioral traits for decision-making, such as date matching, hiring placement, or tele-behavioral services.

### BACKGROUND

The present invention relates to a media or video journal service, and more specifically to media or video journal service which uses short videos recorded numerous times a day for a period of time which can determine biorhythms of the user.

“Match services” to fill a position rely on a candidate's self-perception, which is often insufficient for discerning compatibility for relationships and/or teams.

Dating sites rely on users to build their own profiles by uploading or linking photographs and interests, by setting preferences, and sometimes by creating and answering questions. Some sites additionally require a link to a user's social media account(s). Analytics used to obtain the user's matches rely mostly on interests and preferences of the user.

A third-party company is sometimes used to match job candidates to particular jobs. The third-party company creates video recordings of interviews of potential candidates which are manually edited to create a series of short clips to send to the companies filling jobs. The potential match screening is only based on the third-party company's recruiter's assessment.

Other companies make videos of the hiring manager of the company describing the job that needs to be filled and what they are looking for in a candidate. These videos are posted on the company's website. Any match screening that takes place is on behalf of the candidate “getting a feel” for a hiring manager's management style and priority among the list of desired traits.

The drawbacks of these approaches are that companies are either not using the video at all or they are not maximizing the potential of video to augment the service. These approaches are overly dependent upon self-reporting to create profiles, and assessments lack analytical understanding.

### SUMMARY

According to one embodiment of the present invention, a method of matching users through captured media is disclosed. The method including the steps of: a computer receiving video recordings of a user from different times of day and storing the video recordings with an associated user profile; the computer capturing data from the video recordings through cognitive analysis to identify at least personality traits, emotions, and biorhythms of a user and storing captured data in the user profile; the computer creating a first video journal of compiled video clips extracted from all of the user's video recordings showing the user exhibiting a first identified emotion and storing the first journal in a repository and associated with the user profile; the computer comparing the user profile to other user profiles to obtain a similarity score; and the computer determining other user profiles within a range of the similarity score of the user profile and storing the other user profiles as matches to the user.

According to another embodiment of the present invention a computer program product for matching users through captured media recorded by a computer is disclosed. The computer includes at least one processor, one or more memories, a speaker, a microphone, a video recorder, and one or more computer readable storage media. The computer program product includes a computer readable storage medium having program instructions embodied therewith, the program instructions executable by the computer to perform a method including: receiving, by the computer, video recordings of a user from different times of day and storing the video recordings with an associated user profile; capturing, by the computer, data from the video recordings through cognitive analysis to identify at least personality traits, emotions, and biorhythms of a user and storing captured data in the user profile; creating, by the computer, a first video journal of compiled video clips extracted from all of the user's video recordings showing the user exhibiting a first identified emotion and storing the first journal in a repository and associated with the user profile; comparing, by the computer, the user profile to other user profiles to obtain a similarity score; and determining, by the computer, other user profiles within a range of the similarity score of the user profile and storing the other user profiles as matches to the user.

According to yet another embodiment of the present invention a computer system for matching users through captured media recorded by a device is disclosed. The computer system including a computer comprising at least one processor, a microphone, a speaker, a video recorder, one or more memories, one or more computer readable storage media having program instructions executable by the computer to perform the program instructions including: receiving, by the computer, video recordings of a user from different times of day and storing the video recordings with an associated user profile; capturing, by the computer, data from the video recordings through cognitive analysis to identify at least personality traits, emotions, and biorhythms of a user and storing captured data in the user profile; creating, by the computer, a first video journal of compiled video clips extracted from all of the user's video recordings showing the user exhibiting a first identified emotion and storing the first journal in a repository and associated with the user profile; comparing, by the computer, the user profile to other user profiles to obtain a similarity score; and determining, by the computer, other user profiles within a range of the similarity score of the user profile and storing the other user profiles as matches to the user.

### BRIEF DESCRIPTION OF THE DRAWINGS

FIG. 1 depicts an exemplary diagram of a possible data processing environment in which illustrative embodiments may be implemented.

FIG. 2 illustrates internal and external components of a client computer and a server computer in which illustrative embodiments may be implemented.

FIG. 3 shows a schematic of an example of an interface of a device computer capturing video of a registered user.

FIG. 4 shows a flow diagram of a method of registering a user for a video service.

FIG. 5A shows a schematic of a series of captured video recordings for a first user.

FIG. 5B shows a schematic of a series of captured video recording for a second user which is an example of a match for the first user.

FIG. 6 shows a flow diagram of a method of matching registered users through captured media.

### DETAILED DESCRIPTION

In an embodiment of the present invention, candidate profiles are built based on personality traits and biorhythms. In another embodiment education can also be used to build candidate profiles. A biorhythm is a cyclic pattern of physical, emotional, or mental activity that occurs in the life of a person. The profile data can be used to augment existing self-perception-based profiles. Examples of the video journal service described herein build video journals of candidates by capturing short video segments through the day in a life of the user for days, weeks, and/or months. The collections of the short video segments are used to build the candidate's profile and to find suitable matches for the user personally and professionally.

In another embodiment of the present invention, short video segments are captured through the day in a life of a user for days, weeks, and/or months to build a video journal which can be analyzed to determine biorhythms of the user. The video journal can be compiled to exhibit emotions for a specific time period. The video journal can be shared with other users. The collections of the short video segments are used to build a user profile that can be used by a services that rely on personality or behavioral traits for decision-making, such as date matching, hiring placement, or tele-behavioral services.

For example, a patient can record short videos throughout their day. Examples of the video journal service described herein build video journals exhibiting an emotion by the user and can be shared with another user, for example a medical professional.

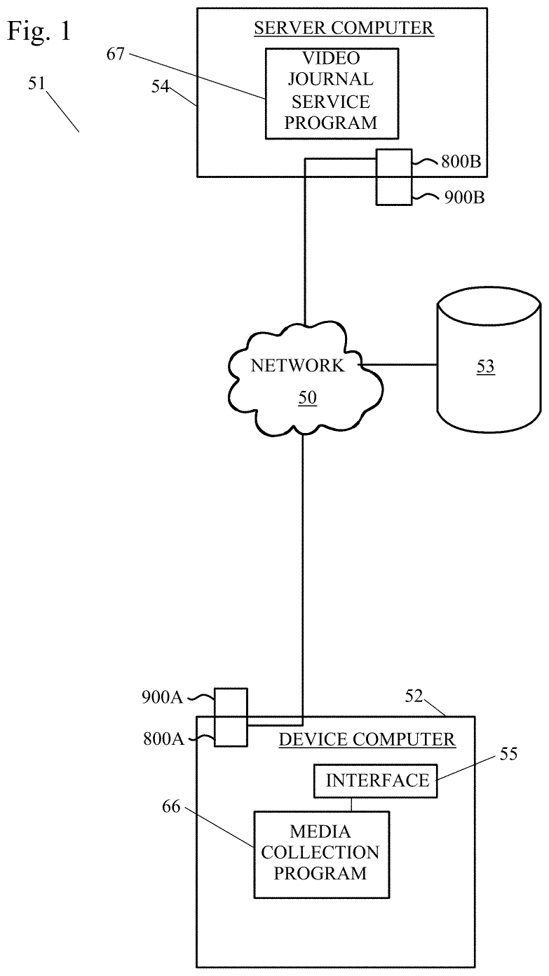

FIG. 1 is an exemplary diagram of a possible data processing environment provided in which illustrative embodiments may be implemented. It should be appreciated that FIG. 1 is only exemplary and is not intended to assert or imply any limitation with regard to the environments in which different embodiments may be implemented. Many modifications to the depicted environments may be made.

Referring to FIG. 1, network data processing system 51 is a network of computers in which illustrative embodiments may be implemented. Network data processing system 51 contains network 50, which is the medium used to provide communication links between various devices and computers connected together within network data processing system 51. Network 50 may include connections, such as wire, wireless communication links, or fiber optic cables.

In the depicted example, device computer 52, a repository 53, and a server computer 54 connect to network 50. In other exemplary embodiments, network data processing system 51 may include additional client or device computers, storage devices or repositories, server computers, and other devices not shown.

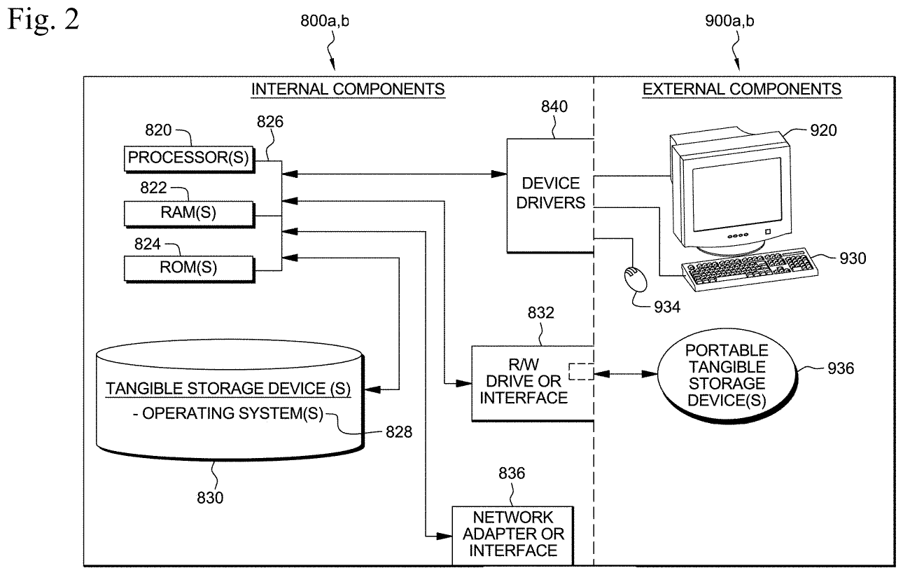

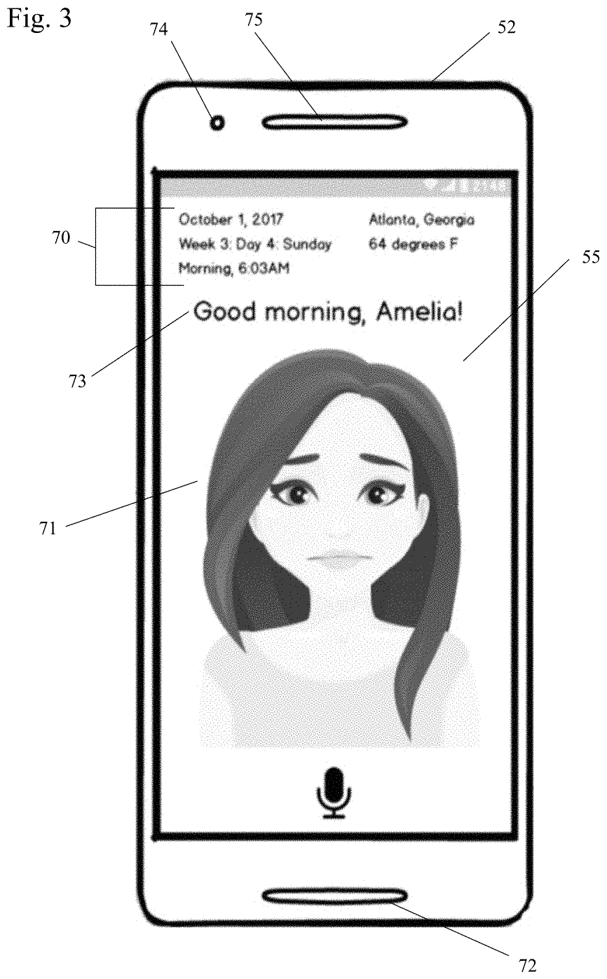

The device computer 52 may contain an interface 55, which may accept commands and data entry from a user. The commands may be regarding video recording, accepting matches, rejecting matches, request for video journals and viewing previously captured video recordings, and accepting notifications. The interface can be, for example, a command line interface, a graphical user interface (GUI), a natural user interface (NUI) or a touch user interface (TUI). The device computer 52 includes a media collection program 66. While not shown, it may be desirable to have the media collection program 66 present on the server computer 54. The device computer 52 includes a set of internal components 800a and a set of external components 900a, further illustrated in FIGS. 2-3.

Server computer 54 includes a set of internal components 800b and a set of external components 900b illustrated in FIG. 2. In the depicted example, server computer 54 provides information, such as boot files, operating system images, and applications to the device computer 52. Server computer 54 can compute the information locally or extract the information from other computers on network 50. The server computer 54 includes a video journal service program 67 in this example and may contain the media collection program 66 in other embodiments.

Repository 53 may contain user profiles, stored video recordings received from registered users, and video journals comprising clips from the stored video recordings.

Program code and programs such as media collection program 66 and video journal service program 67 may be stored on at least one of one or more computer-readable tangible storage devices 830 shown in FIG. 2, on at least one of one or more portable computer-readable tangible storage devices 936 as shown in FIG. 2, or on storage unit 53 connected to network 50, or may be downloaded to a device computer 52 or server computer 54, for use. For example, program code and programs such as media collection program 66 and video journal service program 67 may be stored on at least one of one or more storage devices 830 on server computer 54 and downloaded to device computer 52 over network 50 for use. Alternatively, server computer 54 can be a web server, and the program code, and programs such as media collection program 66 and video journal service program 67 may be stored on at least one of the one or more storage devices 830 on server computer 54 and accessed device computer 52. In other exemplary embodiments, the program code, and programs such as media collection program 66 and video journal service program 67 may be stored on at least one of one or more computer-readable storage devices 830 on device computer 52 or distributed between two or more servers.

In the depicted example, network data processing system 51 is the Internet with network 50 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, network data processing system 51 also may be implemented as a number of different types of networks, such as, for example, an intranet, local area network (LAN), or a wide area network (WAN). FIG. 1 is intended as an example, and not as an architectural limitation, for the different illustrative embodiments.

FIG. 2 illustrates internal and external components of a device computer 52 and server computer 54 in which illustrative embodiments may be implemented. In FIG. 2, a device computer 52 and a server computer 54 include respective sets of internal components 800a, 800b and external components 900a, 900b. Each of the sets of internal components 800a, 800b includes one or more processors 820, one or more computer-readable RAMs 822 and one or more computer-readable ROMs 824 on one or more buses 826, and one or more operating systems 828 and one or more computer-readable tangible storage devices 830. The one or more operating systems 828, media collection program 66, and video journal service program 67 are stored on one or more of the computer-readable tangible storage devices 830 for execution by one or more of the processors 820 via one or more of the RAMs 822 (which typically include cache memory). In the embodiment illustrated in FIG. 2, each of the computer-readable tangible storage devices 830 is a magnetic disk storage device of an internal hard drive. Alternatively, each of the computer-readable tangible storage devices 830 is a semiconductor storage device such as ROM 824, EPROM, flash memory or any other computer-readable tangible storage device that can store a computer program and digital information.

Each set of internal components 800a, 800b also includes a R/W drive or interface 832 to read from and write to one or more portable computer-readable tangible storage devices 936 such as a CD-ROM, DVD, memory stick, magnetic tape, magnetic disk, optical disk or semiconductor storage device. Media collection program 66 and video journal service program 67 can be stored on one or more of the portable computer-readable tangible storage devices 936, read via R/W drive or interface 832 and loaded into hard drive 830.

Each set of internal components 800a, 800b also includes a network adapter or interface 836 such as a TCP/IP adapter card. Media collection program 66 and video journal service program 67 can be downloaded to the device computer 52 and server computer 54 from an external computer via a network (for example, the Internet, a local area network or other, wide area network) and network adapter or interface 836. From the network adapter or interface 836, media collection program 66 and video journal service program 67 are loaded into hard drive 830. Media collection program 66 and video journal service program 67 can be downloaded to the server computer 54 from an external computer via a network (for example, the Internet, a local area network or other, wide area network) and network adapter or interface 836. From the network adapter or interface 836, media collection program 66 and video journal service program 67 are loaded into hard drive 830. The network may comprise copper wires, optical fibers, wireless transmission, routers, firewalls, switches, gateway computers and/or edge servers.

Each of the sets of external components 900a, 900b includes a computer display monitor 920, a keyboard 930, and a computer mouse 934. Each of the sets of internal components 800a, 800b also includes device drivers 840 to interface to computer display monitor 920, keyboard 930 and computer mouse 934. The device drivers 840, R/W drive or interface 832 and network adapter or interface 836 comprise hardware and software (stored in storage device 830 and/or ROM 824).

Media collection program 66 and video journal service program 67 can be written in various programming languages including low-level, high-level, object-oriented or non object-oriented languages. Alternatively, the functions of media collection program 66 and video journal service program 67 can be implemented in whole or in part by computer circuits and other hardware (not shown).

FIG. 3 shows a schematic of an example of an interface of a device computer capturing video of a registered user.

The device computer 52, which in this example is a mobile phone, has a screen or touch user interface 55, a video recorder 74, a microphone 72 for receiving voice recordings and commands from the user, and at least one speaker 75 for playing a prompt or instructions to the user from the media collection program 66. Based on a prompt or notification from the media collection program 66, the user records one's self for a short duration during different times of the day. The media collection program 66 can provide prompts or instructions 73 for what the user should record or say during the recorded video 71 which is recorded via the microphone 72. During the recorded video 71 of the user, information 70 may also be displayed, such as date, time, location derived from a global positioning system (GPS), temperature, and how many days or weeks the user has been subscribed to the media match service. During the recorded video 71, the user can speak into the microphone 72 and the video recorder 74 records the user. In some embodiments, the recorded videos are between 15 seconds and 5 minutes long, although longer video clips may also be used. In particular, in some embodiments, the videos recorded are 15-20 seconds long.

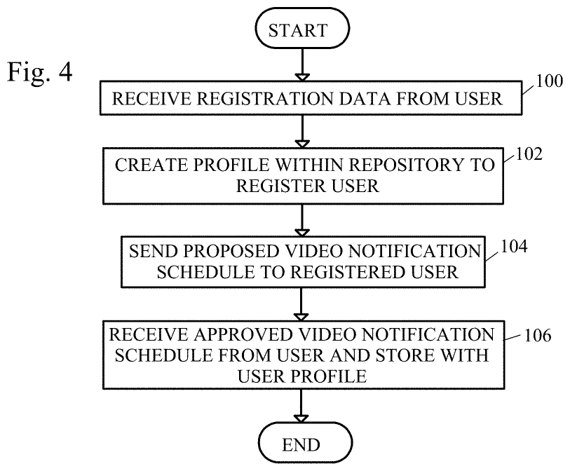

FIG. 4 shows a flow diagram of a method of registering a user for a media match service. In a first step, the video journal service program 67 receives registration information from a user (step 100). The registration information may be received from the media collection program 66 of the device computer 52 via the network 50. The registration information can include name, birth date, contact information, and personal attributes pertinent to the service purpose/function, such as interests, religious and or political preferences. The video journal service program 67 creates a profile within a database to register the user based on the registration information received (step 102). The database may be present on repository 53.

The video journal service program 67 sends a proposed video notification schedule to the user (step 104). The proposed video notification includes a number of times for providing video recordings, times at which to be sent notifications to record a video, and means by which the notifications are to be sent to the user, for example via text message, phone call, calendar appointments, or e-mail. The user can propose a new time at which, or number of times, to provide video recordings to meet the needs of the user.

The video journal service program 67 receives an approved video notification schedule from the user and stores the notification schedule in the user's profile (step 106), and the method ends.

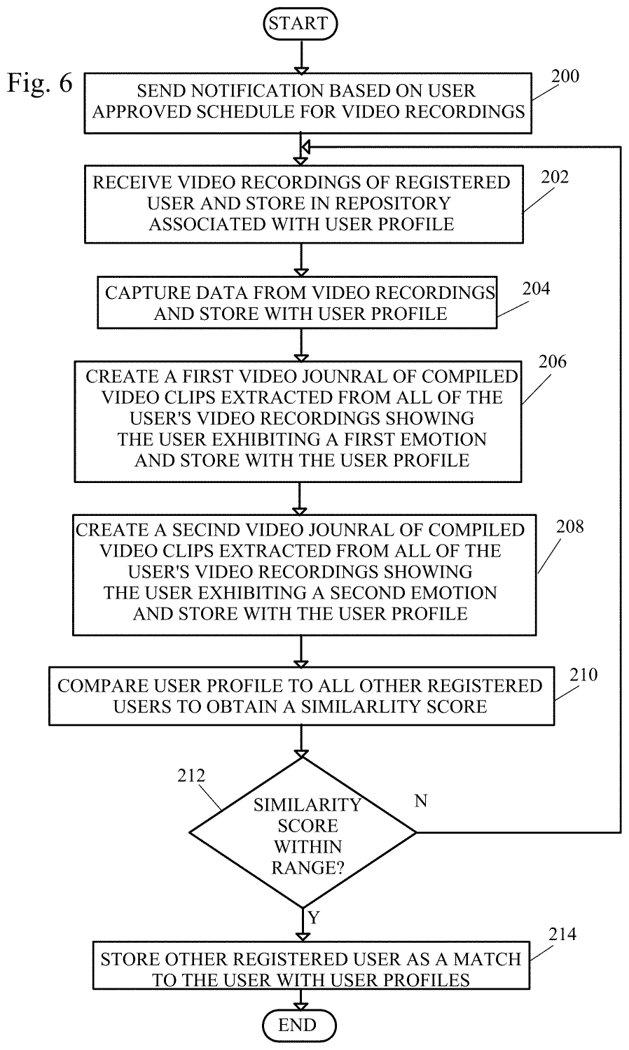

It should be noted that the method of registering the user to create profile of FIG. 4 takes place prior to the method of FIG. 6.

FIG. 6 shows a flow diagram of a method of matching registered users through captured media.

In a first step, the video journal service program 67 sends a notification to the user based on the user approved schedule for video recordings (step 200). As mentioned previously, the notifications can be sent through various means, such as calendar appointments, text messages, phone call, and e-mails.

The video journal service program 67 receives video recordings of the registered user and stores the video recordings associated with the user's profile in a repository, for example repository 53 (step 202). The video recordings can additionally include GPS information and timestamp data. The video recordings are from several different times of day, in some embodiments.

The video journal service program 67 then captures data from the recorded videos and stores the data with the user's profile (step 204). The video journal service program 67 uses cognitive analysis of the user within the video recording, such as speech to text, natural language understanding, tone analyzer, sentiment analysis, visual recognition, and personality insights to determine biorhythms, location, and personality traits through facial expressions, emotions being expressed, and speech spoken by the user. In an alternate embodiment, education may also be determined through linguistics analysis.

By determining at least biorhythms and personality traits from the recorded videos, the self-reported data from the user is significantly decreased. The cognitive analysis of the video recordings can also provide annotations to indicate where certain emotions of the user are being expressed. The annotations can further be used to edit or cut the video clips to just show a specific emotion being expressed. Other means of editing or cutting the video clips can also be used.

The video journal service program 67 then creates a first video journal of compiled video clips cut or extracted from all of the user video recordings showing the user exhibiting a first emotion and the first video journal is stored in the repository associated with the user's profile (step 206). The first emotion may be when the user is happy.

The video journal service program 67 creates a second video journal of compiled video clips cut or extracted from all of the user video recordings showing the user exhibiting a second emotion and the second video journal is stored in the repository associated with the user's profile (step 208). The second emotion may be when the user is sad.

Video journals may be generated for other emotions being emitted by the user.

The first and second video journals are available for the user to view upon request. Alternatively, the video journals can be presented to the user through the media collection program 66.

The video journal service program 67 compares the user profile which now includes biorhythms and personality traits to all other registered users to obtain a similarity score (step 210).

For example, each attribute category has a scoring scheme or weights and each category scoring scheme is independent of the others in some embodiments. Biorhythms are determined through repeating patterns over periods of time. Personality traits can be determined through a traits analysis service, which can utilize Myers-Briggs Type Indicators (MBTI). In an alternate embodiment, education may also be used in the user profile and compared to the other registered users. Education may be determined through linguistics analysis.

If the similarity score is not within a designated range (step 212) the method returns to step 202 of receiving video recordings from the registered user.

If the similarity score is within the designated range (step 212), the video journal service program 67 stores the other registered user as match (step 214) and the method ends.

The designated range for a match of the similarity scores between registered users can be set by the user or can be set by another company looking to fulfill a position.

Optionally, when a match does occur, a video journal of the matched user can be sent to the user to review. The video journal can be the first video journal, the second video journal or a video journal including all of the recordings the user has recorded.

### EXAMPLE 1

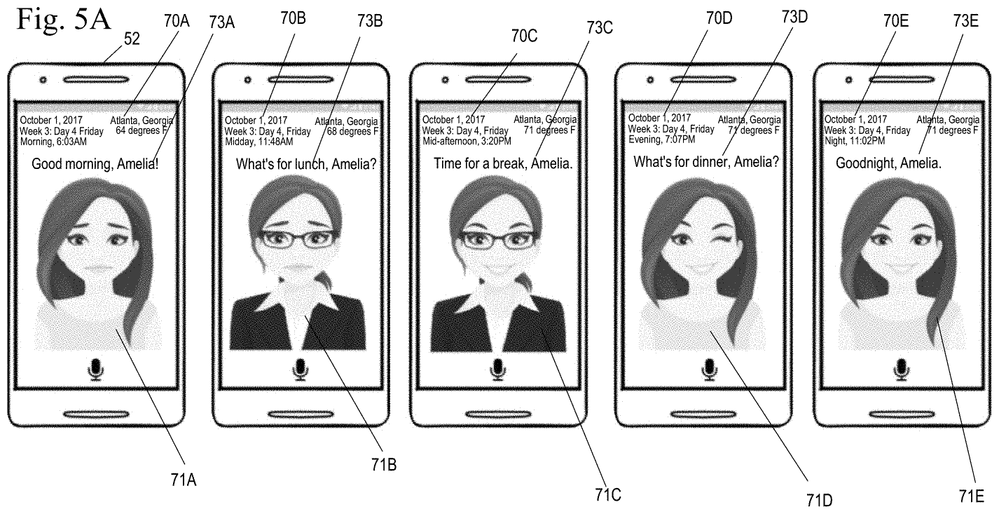

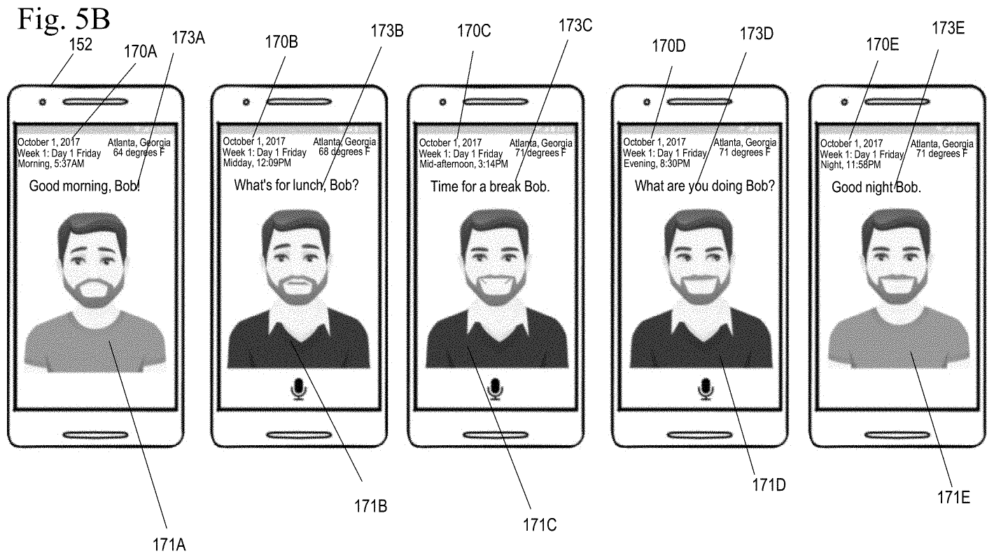

FIG. 5A shows a schematic of a series of captured video recordings for a first user. The video recordings 71A-71E show a series of recordings made by the first user during different times of the day using a mobile phone 52. Each video recording 71A-71E includes associated information 70A-70E such as date, time, location derived from a global positioning system (GPS), temperature, and how many days, weeks the user has been subscribed to the media match service. A different prompt 73A-73E may be provided to the first user for the different video recordings throughout the day.

FIG. 5B shows a schematic of a series of captured video recording for a second user which is an example of a match for the first user. The video recordings 171A-171E show a series of recording made by the second user during different times of the day using a mobile phone 152. Mobile 152 includes the same components as device computer 52. Each video recording 171A-171E includes associated information 170A-170E such as date, time, location derived from a global positioning system (GPS), temperature, and how many days, weeks the user has been subscribed to the media match service. A different prompt 173A-173E may be provided to the first user for the different video recordings throughout the day.

The media match service program 66 can capture data from each of video recordings of the first user and the second user. For example, through cognitive analysis of the first user within video recording 71A and 71B, it could be determined through visual recognition of facial expression and tone analyzer that the first user is not a morning person who wakes up early, without difficulty and is alert and active in the first part of the day. From video recordings 71C-71E it could be determined through visual recognition of facial expression and tone analyzer that the first user is a night owl who likes to be home and feels at the peak physically, emotionally, and intellectually past noon. A similarity score would be calculated to reflect this characteristic.

Similarly, through cognitive analysis of the second user within video recording 171A and 171B, it could be determined through visual recognition of facial expression and tone analyzer that the second user is also not a morning person who wakes up early, without difficulty and is alert and active in the first part of the day. From video recordings 171C-171E it could be determined through visual recognition of facial expression and tone analyzer that the second user is a night owl who likes to be home and feels at the peak physically, emotionally, and intellectually past noon. A similarity score would be calculated to reflect this characteristic.

The similarity scores of the first and second user that are within a range of each other would be a match since the first and second users have a similar biorhythm and similar personalities. Either the first or second user could request a video journal of the other user for viewing. In another embodiment, a user could publically share their video journal(s) or share their video journals with specific other users.

### EXAMPLE 2

A third party consulting company can obtain short videos from a user in which the user describes their technical acumen and business acumen relative to a job or field as well as a compiled video journal of the user based on daily recordings of the user for a specific time period designated by the hiring company. The video journal service program 67 captures data from the recorded videos and stores the data with the user's profile. The video journal service program 67 uses cognitive analysis of the user within the video recording, such as speech to text, natural language understanding, tone analyzer, sentiment analysis, visual recognition, and personality insights to determine biorhythms, location, and personality traits through facial expressions, emotions being expressed, and speech spoken by the user. In an alternate embodiment, education may also be determined through linguistics analysis.

By determining at least biorhythms and personality traits from the recorded videos, the self-reported data from the user is significantly decreased.

The video journal service program 67 then creates a first video journal of compiled video clips cut or extracted from all of the user video recordings showing the user exhibiting a first emotion and the first video journal is stored in the repository associated with the user's profile. The first emotion may be related to personality traits associated with the user.

The video journal service program 67 creates a second video journal of compiled video clips cut or extracted from all of the user video recordings showing the user exhibiting a second emotion and the second video journal is stored in the repository associated with the user's profile. The second emotion may be related to personality traits associated with the user.

The video journal service program 67 compares the user profile which now includes biorhythms and personality traits to all other registered users or candidates looking to fulfill the position with the hiring company to obtain a similarity score.

For example, each attribute category has a scoring scheme or weights and each category scoring scheme is independent in some embodiments. Biorhythms are determined through repeating patterns over periods of time. Personality traits can be determined through a traits analysis service, which can utilize Myers-Briggs Type Indicators (MBTI). In an alternate embodiment, education may also be used in the user profile and compared to the other registered users. Education may be determined through linguistics analysis or other input.

Based on similarity scores, the hiring company can choose and build teams of users that have complementary biorhythms and personalities.

In another embodiment, the media match service may just analyze the videos received from the user to capture data and determine a user's biorhythms and personality traits.

### EXAMPLE 3

A patient can be the user and can record short videos throughout their day. The video journal service program 67 builds video journals exhibiting an emotion by the user and can be shared with another user, for example a medical professional. The medical professional can use this service to aid in monitoring a patient within a specific time period.

Short video segments are captured through the day in a life of a patient for days, weeks, and/or months to build a video journal which can be analyzed to determine biorhythms of the patient. The video journal can be compiled to exhibit emotions for a specific time period. The video journal can be shared with other users, such as medical professionals, caregivers or family members.

In this embodiment, the video journal service program 67 sends a notification to the patient based on the patient approved schedule for video recordings. As mentioned previously, the notifications can be sent through various means, such as calendar appointments, text messages, phone call, and e-mails.

The video journal service program 67 receives video recordings of the registered patient and stores the video recordings associated with the patient's profile in a repository, for example repository 53. The video recordings can additionally include GPS information and timestamp data. The video recordings are from several different times of day, in some embodiments.

The video journal service program 67 then captures data from the recorded videos and stores the data with the patient's profile. The video journal service program 67 uses cognitive analysis of the patient within the video recording, such as speech to text, natural language understanding, tone analyzer, sentiment analysis, visual recognition, and personality insights to determine at least biorhythms, location, and personality traits through facial expressions, emotions being expressed, and speech spoken by the patient.

By determining at least biorhythms and personality traits from the recorded videos, the self-reported data from the patient is significantly decreased. The cognitive analysis of the video recordings can also provide annotations to indicate where certain emotions of the patient are being expressed. The annotations can further be used to edit or cut the video clips to just show a specific emotion being expressed. Other means of editing or cutting the video clips can also be used.

The video journal service program 67 then creates a first video journal of compiled video clips cut or extracted from all of the patient video recordings showing the patient exhibiting a first emotion and the first video journal is stored in the repository associated with the patient's profile. The first emotion may be when the patient is happy.

The video journal service program 67 creates a second video journal of compiled video clips cut or extracted from all of the patient video recordings showing the patient exhibiting a second emotion and the second video journal is stored in the repository associated with the patient's profile. The second emotion may be when the patient is sad.

Video journals may be generated for other emotions being emitted by the patient.

The first and second video journals can be available for the patient to view upon request. Alternatively, the video journals can be presented to the patient through the media collection program 66.

The first and second video journals are shared with another user, such as a medical professional, caregiver or family member for review.

The method/system of embodiments of the present invention enables the computer to use video journals to perform a new functionality to match users without subjective human user input, which focuses on a improving video journaling technology and match services technology. The method/system of embodiments of the present invention maximizes the potential of videos associated with candidates or other users for a match service without relying on self-reporting of the users in profile creation and assessments lacking analytical understanding.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method of matching users through captured media comprising the steps of:
a computer receiving video recordings of a user from different times of day and storing the video recordings with an associated user profile;
the computer capturing data from the video recordings through cognitive analysis to identify at least personality traits, emotions, and biorhythms of a user and storing captured data in the user profile;
the computer creating a first video journal of compiled video clips extracted from all of the user's video recordings showing the user exhibiting a first identified emotion and storing the first journal in a repository associated with the user profile;
the computer comparing the user profile to other user profiles to obtain a similarity score; and
the computer determining other user profiles within a range of the similarity score of the user profile and storing the other user profiles as matches to the user.

2. The method of claim 1, wherein cognitive analysis further identifies education of the user.

3. The method of claim 1, wherein the video recordings are between 15 seconds and 5 minutes long.

4. The method of claim 1, wherein the video recordings are between 15 and 20 seconds long.

5. The method of claim 1, wherein the video recordings include location data and timestamp data.

6. The method of claim 1, further comprising the step of the computer annotating portions of the video recordings for specific emotions emitted by the user, such that the video recordings can be cut based on the annotations to compile a video journal.

7. The method of claim 1, further comprising the step of the computer creating a second video journal of compiled video clips extracted from all of the user's video recordings showing the user exhibiting a second identified emotion and storing the second journal in a repository associated with the user profile.

8. The method of claim 7, wherein the first identified emotion is happiness and the second identified emotion is sadness.

9. The method of claim 1, further comprising the step of the computer sending the first video journal of the user to other users identified as matching the user profile.

10. A computer program product for matching users through captured media recorded by a computer comprising at least one processor, one or more memories, a speaker, a microphone, a video recorder, one or more computer readable storage media, the computer program product comprising a computer readable storage medium having program instructions embodied therewith, the program instructions executable by the computer to perform a method comprising:
receiving, by the computer, video recordings of a user from different times of day and storing the video recordings with an associated user profile;
capturing, by the computer, data from the video recordings through cognitive analysis to identify at least personality traits, emotions, and biorhythms of a user;
storing, by the computer, captured data in the user profile;
creating, by the computer, a first video journal of compiled video clips extracted from all of the user's video recordings showing the user exhibiting a first identified emotion;
storing, by the computer, the first journal in a repository associated with the user profile;
comparing, by the computer, the user profile to other user profiles to obtain a similarity score; and
determining, by the computer, other user profiles within a range of the similarity score of the user profile and storing the other user profiles as matches to the user.

11. The computer program product of claim 10, wherein the video recordings are between 15 and 20 seconds long.

12. The computer program product of claim 10, wherein the video recordings include location data and timestamp data.

13. The computer program product of claim 10, further comprising the program instructions creating, by the computer, a second video journal of compiled video clips extracted from all of the user's video recordings showing the user exhibiting a second identified emotion and storing the second journal in a repository and associated with the user profile.

14. The computer program product of claim 13, wherein the first identified emotion is happiness and the second identified emotion is sadness.

15. The computer program product of claim 10, further comprising the program instructions of sending, by the computer, the first video journal of the user to other users identified as matching the user profile.

16. A computer system for matching users through captured media recorded by a device comprising a computer comprising at least one processor, a microphone, a speaker, a video recorder, one or more memories, one or more computer readable storage media having program instructions executable by the computer to perform the program instructions comprising:
receiving, by the computer, video recordings of a user from different times of day and storing the video recordings with an associated user profile;
capturing, by the computer, data from the video recordings through cognitive analysis to identify at least personality traits, emotions, and biorhythms of a user;
storing, by the computer, captured data in the user profile;
creating, by the computer, a first video journal of compiled video clips extracted from all of the user's video recordings showing the user exhibiting a first identified emotion;
storing, by the computer, the first journal in a repository associated with the user profile;
comparing, by the computer, the user profile to other user profiles to obtain a similarity score; and
determining, by the computer, other user profiles within a range of the similarity score of the user profile and storing the other user profiles as matches to the user.

17. The computer system of claim 16, wherein the video recordings are between 15 and 20 seconds long.

18. The computer system of claim 16, further comprising the program instructions creating, by the computer, a second video journal of compiled video clips extracted from all of the user's video recordings showing the user exhibiting a second identified emotion and storing the second journal in a repository associated with the user profile.

19. The computer system of claim 18, wherein the first identified emotion is happiness and the second identified emotion is sadness.

20. The computer system of claim 16, further comprising the program instructions of sending, by the computer, the first video journal of the user to other users identified as matching the user profile.
